(19) United States
(12) Patent Application Publication
(10) Pub. No.: US 2012/0020941 A1
(43) Pub. Date: Jan. 26, 2012

(54) USE OF BLOOD GROUP STATUS III

(75) Inventors: Pirjo WACKLIN, Helsinki (FI); Jaana MÄTTÖ, Helsinki (FI); Harri MÄKIVUOKKO, Kirkkonummi (FI); Jukka PARTANEN, Helsinki (FI)

(73) Assignee: SUOMEN PUNAINEN RISTI VERIPALVELU, Helsinki (FI)

(21) Appl. No.: 12/843,409

(22) Filed: Jul. 26, 2010

(51) Int. Cl. A61K 35/74; A61P 31/00; A61P 3/00; C12Q 1/68; A61P 1/00

(52) U.S. Cl. 424/93.41; 424/93.4; 435/6.15

(57) ABSTRACT

Provided is a microbial composition which is tailored based on the spectrum of microbes found more frequently from the intestine of non-secretor individuals than from the intestine of secretor individuals. Further provided is a method of tailoring a microbial composition based on the spectrum of microbes found more frequently from the intestine of non-secretor individuals than from that of secretor blood group status. Further provided is a use of the secretor status of an individual as a criterion for microbial supplementation tailored based on the differences in the spectra of microbes found between secretor and non-secretor individuals.

### FIELD OF THE INVENTION

The present invention relates to a microbial composition which is tailored based on the spectrum of microbes found more frequently from the intestine of non-secretor individuals than from the intestine of secretor individuals. The present invention further relates to a method of tailoring a microbial composition based on the spectrum of microbes found more frequently from the intestine of non-secretor individuals than from that of secretor blood group status. The present invention relates to use of the secretor status of an individual as a criterion for microbial supplementation tailored based on the differences in the spectra of microbes found between secretor and non-secretor individuals. The present invention relates also to method of assessing the need of an individual for the tailored microbial supplementation by determining the secretory status of the individual. Also, the invention relates to a method of treating and/or preventing disorders related to unbalanced mucosal microbiota in an individual.

### BACKGROUND OF THE INVENTION

Human intestinal tract is colonised with over 500 bacterial species, whose total number can exceed trillions of microbial cells in the colon. This microbiota in the large intestine is mainly composed of Firmicutes and Bacteroides phyla, which make up over 75% and 16% of total microbes in the gut (Eckburg et al., 2005, Science 308(5728):1635-8, Tap et al., 2009, Environ Microbiol 11(10):2574-84). Within Firmicutes phyla, Clostridium and its close relatives dominate with Clostridium leptum group (Clostridium cluster IV) and Clostridium coccoides group (Clostridium cluster XIVa) are the most prevalent groups (Tap et al. 2009). Bacteroides species found in the gut mainly belong to B. fragilis group. In spite of low diversity at the microbial phyla level, the gut microbiota composition among individuals is highly variable at species and strain level. In 17 human faecal samples, only 66 OTUs (“Operational Taxonomic Units”) of the 3180 detected were present in more than 50% of the individuals, creating so-called core microbiota (Tap et al. 2009). The core microbiota consisted mainly species of Bacteroides and clostridia; in addition, one Bifidobacterium spp and one Coprobacillus spp. were included in the core.

The microbiota has an important role in human health. It contributes to the maturation of the gut tissue, to host nutrition, pathogen resistance, epithelial cell proliferation, host energy metabolism and immune response (e.g. Dethlefsen et al., 2006, Trends Ecol Evol 21(9):517-23; Round and Mazmanian, 2009, Nat Rev Immunol 9(5):313-23). An altered composition and diversity of gut microbiota have been associated to several diseases (Round and Mazmanian, 2009), such as inflammatory bowel disease, IBD (Sokol et al., 2008, Proc Natl Acad Sci USA, 105(43):16731-6), irritable bowel syndrome (Mättö et al. 2005, FEMS Immunol Med Microbiol 43(2):213-22.), rheumatoid arthritis (Vaahtovuo et al., 2008, J Rheumatol 35(8):1500-5), atopic eczema (Kalliomaki and Isolauri. 2003 Curr Opin Allergy Clin Immunol 3: 15-20), asthma (Björksten 1999 Curr Drug Targets Inflamm Allergy 4: 599-604) type 1 diabetes (Wen et al., 2008, Nature 455(7216):1109-13). Little is known, however, which species mediate beneficial responses. A decrease in the number of Faecalibacterium prausnitzii, a well-studied member of the C. leptum group, has been observed in IBD and evidence indicates that F. prausnitzii has anti-inflammaroty effects in vitro and in vivo (Sokol et al. 2008).

The role of host genes on composition of gut microbes has been supported by twin studies, which showed that monozygotic twins have more similar gut microbiota than dizygotic twins or unrelated persons (Zoetendal et al., 2001, Microbial Ecology in Health and Disease 13(3):129-34). However, little is known which genes determine or regulate the microbial composition. Some gut microbes e.g. Helicobacter pylori and pathogenic species of bacteria and viruses have shown to use ABO blood group antigens as adhesion reseptors (Boren et al. Science 1993, 262, 1892-1895). Some microbes e.g. Bifidobacteria and Bacteroides thetaiotaomicron are also able to utilize blood group antigens or glycans found in ABO and Lewis antigens.

The ABO blood group antigens are not present in the mucus of all individuals. These individuals, said to have the ‘non-secretor’ blood group, do not have the functional FUT2 gene needed in the synthesis of secreted blood group antigens (Henry et al., Vox Sang 1995; 69(3):166-82). Hence, they do not have ABO antigens in their secretions and mucosa while those with blood group ‘secretor’ have the antigens. In most populations, the frequency of non-secretor individuals is substantially lower than that of secretor status; about 15-26% of Scandinavians are non-secretors (Eriksson et al. Ann Hum Biol. May-June 1986; 13(3):273-85). The secretor/non-secretor status can be regarded as a normal blood group system and the phenotype can be determined using standard blood banking protocols (Henry et al. 1995). The genotype, that is, the major mutation in the FUT2 gene causing the non-secretor (NSS) phenotype in the European populations (Silva et al. Glycoconj 2010; 27:61-8) has been identified. Non-secretor phenotype has been demonstrated to be genetically associated for example, with an increased risk for Crohn's disease (McGovern et al. Hum Molec Genet 2010 Advance Access Published Jun. 22, 2010), with high vitamin B12 levels in the blood (Tanaka et al Am J Hum Genet 2009; 84:477-482), with resistance to Norovirus infection (Thorven et al J Virol 2005; 79: 15351-15355), with susceptibility to HI virus infection (Ali et al 2000, J Infect Dis 181: 737-739), with experimental vaginal candidiasis (Hurd and Domino Infection Immunit 2004; 72: 4279-4281), with an increased risk for asthma (Ronchetti et al. Eur Respir J 2001; 17: 1236-1238), with urinary tract infections (Sheinfeld et al N Engl J Med 1989; 320: 773-777), and with an animal hemorrhagic disease virus (Guillon et al. Glycobiology 2009; 19: 21-28).

The beneficial effects of certain microbial species/strains on maintaining or even improving of gut balance and growing evidence of their health effects on intestinal inflammatory diseases have caused a great interest on modulation of gut microbiota; and recently also on modulation of microbiota of other tissues such as oral, vaginal or skin. Gut microbiota can be modulated by taking probiotics, which currently belong mainly to Bifidobacteria and Lactobacillus genera.

Many probiotic supplements and products currently on the market are ineffective in promoting the desired health effects among most individuals. Thus, there is a continuous need for microbial and/or probiotic products that are able to mediate the health effects of the microbes more efficiently.

### BRIEF DESCRIPTION OF THE INVENTION

The present invention is based on the finding that individuals with non-secretor blood group status showed marked differences in their gut microbial composition in comparison to secretor individuals. Specifically, occurrence or abundance of certain Bacteroides and Clostridium leptum group genotypes, as defined using the method of Denaturating Gradient Gel Electrophoresis (DGGE), were higher in non-secretor individuls than secretor individuals.

The genotypes were:

band positions 25.30%, 26.40%, 50.40% and 56.80% as defined by universal-DGGE analysis;

band position 60.0% as defined by Eubacterium rectale-Clostridium coccoides-group (EREC)-DGGE analysis;

band positions 4.80%, 10.20%, 23.80%, 38.70%, and 41.10% as defined by Bacteroides-DGGE analysis;

and

band positions 32.80%, 36.10%, 43.00%, 73.30%, 79.10%, 85.00%, and 91.80% as defined by Clostridium leptum-DGGE.

Further, secretor/non-secretor status was shown to determine the diversity of Lactobacillus in the gut of an individual.

Thus, the non-secretor blood group status was found to be a host genotype, which determines the composition of intestinal microbes in man. This finding can be used as a basis for targeted modulation of intestinal microbial population tailored according to non-secretor/secretor status of an individual. The invention describes which particular microbes should be enriched in a microbial and/or probiotic supplement or composition to improve the responsiveness and/or effect of the product. This tailoring or optimising or potentiating can be done to an existing microbial, probiotic and/or synbiotic product, or to a microbial strain not currently used as a probiotic.

Thus, an object of the present invention is a microbial composition which is tailored based on the spectrum of microbes found more frequently from the intestine of the non-secretor individuals than from the intestine of secretor individuals. An other object of the invention is use of the secretor blood group status of an individual in assessing the need for tailored microbial supplementation, i.e., as a criterion for microbial supplementation tailored based on the differences in the spectra of microbes found between secretor and non-secretor individuals. The present invention relates also to method of assessing the need of an individual for microbial supplementation by determining the secretory status of the individual. In addition, the invention relates to methods for treating and/or preventing disorders related to unbalanced mucosal microbiota and/or having FUT2 gene as a susceptible factor by administering to an individual an effective amount of the microbial composition of the present invention. Further, the invention relates to a method for treating and/or preventing inflammatory bowel disease and/or urogenital infections and/or low levels of vitamin B12 in an individual by administering to the individual an effective amount of the microbial composition of the present invention.

Also, an object of the invention is the use of prebiotics, molecular compounds or additional supportive microbial strains, to increase the number of, and/or to augment the growth and/or functionality of microbes in the intestine.

A further object of the present invention is a use of the secretor blood group status of an individual in estimating a dose of microbial supplementation needed for a desired effect.

The objects of the invention are achieved by the compositions, methods and uses set forth in the independent claims. Preferred embodiments of the invention are described in the dependent claims.

Other objects, details and advantages of the present invention will become apparent from the following drawings, detailed description and examples.

### DESCRIPTION OF THE DRAWING

FIG. 1 shows the richness, that is, the number of DGGE bands or genotypes detected in Lactobacillus-DGGE and the Simpson diversity index in the samples studied. The non-secretors had a lower number of Lactobacillus genotypes than secretors and a lower Simpson diversity index; the significance in the difference between non-secretor (NSS, n=6) and secretor (SS, n=49) samples was evaluated by ANOVA.

### DETAILED DESCRIPTION OF THE INVENTION

The present invention is based on the finding that a blood group system, secretor/non-secretor status, determines the spectrum or composition of microbial species and/or strains found in the human gut, especially in the intestine. Individuals with non-secretor blood group status had marked differences in their gut microbial composition as compared to individuals with secretor status. According to the present invention, the blood group system secretor/non-secretor is a major genetic factor in the host determining the variation in the microbiota. The secretor/non-secretor status can be regarded as a normal blood group system and the phenotype can be determined using standard blood banking protocols. The genotype, that is, the mutation in the FUT2 gene causing the NSS phenotype can be detected by various standard DNA-based techniques, such as allele-specific PCR amplification, sequencing, or using oligonucleotide probes, well-known in the art. The gut microbiota has an important role in human health; importantly, an altered composition and/or altered diversity of gut microbiota have been associated to several diseases.

According to the present invention, occurrence or abundance (i.e. band intensity) of certain genotypes of Bacteroides and Clostridium leptum group were higher in non-secretor individuals than in secretor individuals. Further, individuals with non-secretor blood group have a reduced amount and/or diversity of Lactobacillus in their intestinal bacterial population. This finding can be used as a basis for targeted modulation of the Lactobacillus population in the non-secretor individuals and as a criterion for Lactobacillus enriched probiotic supplementation.

Denaturating Gradient Gel Electrophoresis, DGGE, is a method of choice to detect differences in spectrum or abundance of different bacterial genotypes. In the method, specific PCR primers are designed so that in each experimental setting, only the desired bacterial group or groups are analysed. The differences in band positions and/or their occurrence and/or intensity indicate differences in bacterial compositions between faecal samples. Base composition of the PCR amplified fragment determinates the melting and, thus the mobility of the fragment in the denaturing gradient in gel. The final position of the fragment in gel is consequently specified by the DNA sequence of the fragment, the applied denaturing gradient and the electrophoresis running conditions. The optimised running conditions and denaturing gradient of the gels for the bacterial groups used in this invention are shown in Table 2. The position of each fragment, the “band position”, between different gel runs are normalised by using standards. The band position is indicated relative to length of the gel, the top being 0% and the bottom edge being 100%. The standards used were composed of PCR amplified fragments of the relevant strains belonging to each bacterial group as described in Table 2.

The term bacterial genotype refers to those strains having the same “band position” in the relevant DGGE analysis. Each genotype or a group of closely-related genotypes can be presented as a “band position”. In the present invention, each band position refers to the band positions of the given %-value+/−1% unit, i.e. 25.30% refers to any value between 24.30% and 26.30%, when analysed using the methodology described above. It is noted than depending on the exact conditions the nominant %-value can vary; the relative position of the band to the relevant standard is important. According to the invention, the following bacterial genotypes had a higher abundance and/or higher band intensity in the gut microbiota of non-secretors than in that of secretors:

band positions 25.30%, 26.40%, 50.40% and 56.80% as defined by universal-DGGE analysis;

band position 60.0% as defined by Eubacterium rectale-Clostridium coccoides-group (EREC)-DGGE analysis;

band positions 4.80%, 10.20%, 23.80%, 38.70%, and 41.10% as defined by Bacteroides-DGGE analysis;

and

band positions 32.80%, 36.10%, 43.00%, 73.30%, 79.10%, 85.00%, and 91.80% as defined by Clostridium leptum-DGGE analysis.

In addition, the following microbial genotypes had a higher frequency or occurrence in samples from non-secretors than from secretors:

band position 56.80% as defined by universal-DGGE analysis;

band position 60.0% as defined by Eubacterium rectale-Clostridium coccoides-group (EREC)-DGGE analysis; and

band position 23.80% as defined by Bacteroides-DGGE analysis.

The above mentioned genotypes are examples of genotypes here referred to as “genotypes typical to individuals” with secretor or non-secretor phenotype. It is of note that as the secretor/non-secretor trait, that is the expression of ABO structures in mucosa, can be identified in all mucosal tissues, the invention is relevant to all mucosal tissues of an individual and not restricted to the gut or faecal samples.

The present invention provides means for the use of secretor status for tailoring probiotic supplements optimized according to non-secretor (NSS) and secretor (SS) genotype of the host. Optimization is based on the rationale that according to the present invention, certain bacterial genotypes are essentially missing or their proportion of the entire gut microbiota is lower in an individual or host having secretor genotype than in non-secretor genotype. The probiotic preparation or product can be tailored so that it contains higher amounts or proportions of those bacterial genotypes or strains that are known to have altered abundances and whose increase in number is desired.

In an embodiment of the invention, the microbial composition comprises at least one of the strains having any of the following genotypes:

band position 25.30%, 26.40%, 50.40% or 56.80% as defined by universal-DGGE analysis; or

band position 60.0% as defined by Eubacterium rectale-Clostridium coccoides-group (EREC)-DGGE analysis; or

band positions 4.80%, 10.20%, 23.80%, 38.70%, or 41.10% as defined by Bacteroides-DGGE analysis; or

band position 32.80%, 36.10%, 43.00%, 73.30%, 79.10%, 85.00%, or 91.80% as defined by Clostridium leptum-DGGE analysis.

In another embodiment, the microbial composition comprises two or more of the strains specified above. In a further embodiment, the desired microbial strains belong to the Clostridium leptum group. In another further embodiment, the microbial composition is enriched with Lactobacillus.

The microbial preparation according to the present invention is targeted, for example, to a relief of symptoms and/or to the therapy of diseases in which gut microbiota plays an important role, such as inflammatory bowel disease, IBD (Sokol et al. 2008.), irritable bowel syndrome (Matto et al. 2005.), rheumatoid arthritis (Vaahtovuo et al. 2008), atopic eczema (Kalliomaki et al. 2003), asthma (Björksten, 1999) and type 1 diabetes (Wen et al. 2008). In one embodiment, the target is general immunomodulation, for example, induction of regulatory T lymphocytes (Round and Mazmanian PNAS doi/10.1073/pnas.0909122107), which are known to induce immunotolerance in organ and stem cell transplantations or to suppress the immune response.

In one embodiment of the invention, the preparation is used, for example, in a relief of symptoms and/or in the therapy of inflammatory bowel disease and other immune system related disorders of the gut.

In another embodiment of the invention, the secretor/non-secretor status can be used to augment the stabilisation of mucosal microbiota composition of an individual after disorders or treatments known to disturb the balance of mucosal microbiota. Examples of these comprise treatments with strong antibiotics, irradiation or cytotoxic therapies related to cancer treatments or bone marrow transplantation and/or gastroenterological infections by e.g. Noro-virus or Helicobacter. The present invention is further targeted to treatment of diseases or traits, having the FUT2 gene (i.e. the secretor blood group status) as a genetic susceptibility factor. These comprise, just to give examples, low levels of vitamin B12 in the blood, various clinical forms of inflammatory bowel disease, urinary tract infections, vaginal candidiasis, Noro- and HI-virus infections and infections by hemorrhagic viruses. It is likely that a higher number of diseases will be identified in the future by screening the FUT2 locus. Probiotic treatments typically are used to direct or change the microbiological balance in the gut toward more healthy one, or toward the microbial spectrum “typical to individuals” with the non-susceptible FUT2 genotype. The present invention is particularly related to treatments directed to individuals with the non-secretor status. Individuals with the non-secretor phenotype typically require higher dosages and/or preparations with more diverse microbial strains than secretors. Thus, the present invention relates also to use of the secretor/non-secretor status of an individual to augment the stabilisation of mucosal microbiota composition in disorders related to, or after treatments leading to unbalance of mucosal microbiota. The present invention also relates to a method for treating and/or preventing disorders or diseases related to unbalanced mucosal microbiota in an individual by administering to the individual an effective amount of the microbial composition of the present invention. The present invention further relates to a method for treating and/or preventing disorders or diseases having FUT2 gene as a susceptible factor in an individual by administering to the individual an effective amount of the microbial composition of the present invention. In addition, the present invention relates to a method for treating and/or preventing inflammatory bowel disease, urogenital infections and/or low levels of vitamin B12 in an individual by administering to the individual an effective amount of the microbial composition of the present invention.

The present invention also relates to a method of identifying an individual at risk for suffering from a disorder related to unbalance of mucosal microbiota, such as a gastrointestinal disorder, an urogenital infection and/or low levels of vitamin B12 by determining the secretory status of said individual.

In one embodiment, the microbial preparation is not orally administered but is a solution or ‘salva’ which is directly administered onto the target mucosal tissue. Examples of this embodiment are disorders of gingival or vaginal tissues.

The present invention further relates to a use of the secretor/non-secretor status of an individual in estimating a dose of bacterial supplementation needed for a desired effect.

In one embodiment, the invention is related to microbial or probiotic composition targeted to elderly individuals for supporting the maintenance of balanced microbiota in the gut and/or some other mucosal, such as oral, vaginal or skin tissue. In another embodiment, the invention is related to microbial or probiotic composition targeted to infants for stabilisation of the microbiota in the gut and/or some other mucosal tissue. Limited repertoire of commensal microbes typical to infants confers them susceptible for infections; optimisation of the composition according to the present invention increases the efficacy of the treatment. The treatment can be either prophylactic before an infection for individuals, e.g. elderly or infants, with a high infection risk (i.e, probiotic type), or therapeutic during the infection.

The present invention also provides means for improving responsiveness and/or effect of the microbial and/or probiotic product. Not all individuals are responsive for current probiotic products; a tailored composition enriched with microbial strains which according to the present invention have a better ability to stay alive and grow in the gut or other mucosal tissue improves responsiveness.

Severe disturbances in the gut microbiota can be a result of treatments related to e.g. cancer therapy, haematopoietic stem cell transplantation, or use of antibiotics. The present invention relates to the use of secretor/non-secretor status in estimating the most effective way for stabilisation of the microbiota. Stabilisation can be achieved most effectively by probiotic products tailored according to the present invention.

The present invention provides a novel and effective method for screening and identification of novel probiotic strains. In one embodiment, the NSS/SS genotype forms the basis for the selection of the most efficient source of the faecal samples, the starting point for identification of suitable probiotics. Faecal samples from individuals with non-secretor status can be used for isolating efficiently those bacterial strains more abundant in non-secretor genotype. The fact that these strains, e.g. those belonging to C. leptum or B. fragilis group, are frequent in the microbiota of hosts with NSS genotype indicates that they obviously are particularly viable in the gut of NSS hosts. A good colonization ability and viability in the gut are essential features for a probiotic. The invention can be applied in the similar way when other mucosal tissues than the gut are considered as a target.

In a preferred embodiment of the present invention, determination of the secretor/non-secretor status and use of the result to consequently predict the bacterial spectrum of an individual is used to optimize faecal transplantation. This can be done as the only test or in combination with an actual analysis of microbiota composition. The result can be used as a criterion for choosing a donor for faecal transplantation. Bacteria derived from the faecal transplant from a donor representing the same secretor/non-secretor type with the recipient are likely to have a better colonisation ability and efficacy than those derived from a mismatched donor. Faecal transplantation can be used for a therapy in severe Clostridium difficile infections (MacConnachie et al. QJM 2009, 102(11), 781-4); the present invention can improve the efficacy of the treatment. The efficacy can be further improved by giving a secretor/non-secretor matched bacterial preparation post-transplantation in order to improve the stabilisation of the gut microbiota of the recipient. The preparation can contain the spectrum of bacteria found commonly in samples classified according to sectretor/non-secretor status and can be produced e.g. as a fresh, frozen pellet or freeze-dried product formulation. In addition to Clostridium difficile infection, faecal transplantation once optimised according to the present invention can be used to stabilise gut microbiota in many other disorders related to or resulting to severe disturbances in gut microbiota, for example, diseases requiring intensive antibiotic treatments, chemotherapy or total body irradiation before bone marrow transplantation.

In an embodiment, the secretor/non-secretor status is used, together with standard analyses of microbial composition in a sample, in estimating whether microbial composition in a particular mucosal tissue, such as the gut of an individual is in balance. The genotype can be determined in vitro from the blood or saliva sample of the host and the microbial composition from the mucosal or faecal samples using standard methods, well known in the art. Host secretor/non-secretor genotype together with the standard analysis of microbial spectrum, provides a more reliable estimate of the balance than the analysis of the mucosal or faecal sample alone, because the genotype partially determines the assumed, normal composition. This result can be used to estimate the need by an individual for probiotic supplementation in disorders assumed or known to be related to variation in the microbiota. The result can also be used to reduce infection risk. Non-secretors are known to be more vulnerable to infections (Blackwell, C. C. 1989. FEMS Microbiology Immunology 47, 341-350). A balanced and diverse population of beneficial commensal gut microbes, achieved or augmented by probiotics tailored according to the present invention, is therefore particularly important for non-secretors.

The term ‘probiotic’ here refers to any bacterial species, strain or their combinations, with health supportive effects, not limited to currently accepted strains or to intestinal effects. The probiotic as defined here may be applied also by other routes than by ingestion, e.g. by applying directly to desired tissue.

The term ‘prebiotic’ here refers to any compound, nutrient, or additional microbe applied as a single additive or as a mixture, together with probiotics or without probiotics, in order to augment a desired probiotic health effect or to stimulate the growth and activity of those microbes in the mucous tissue, such as digestive system, which are assumed to be beneficial to the health of the host body.

The terms “microbial” and “bacterial” here are used as synonyms and refer to any bacterial or other microbial species, strains or their combinations, with health supportive effects, not limited to strains currently accepted as probiotics.

The terms “microbial composition or microbial product” here refer to a microbial preparation and a probiotic or prebiotic product, including those applied by other routes than the traditional ingested probiotic, e.g. applied directly onto mucosal tissues such as skin or uro-genital tract, or a product for faecal transplant.

The term “ tailored” refers to targeted modulation based on the secretor/non-secretor genotype of an individual.

The probiotic compositions and supplements so designed may have beneficial effects on the health and/or well-being of a human and may be formulated into a functional food product or a nutritional supplement as well as a capsule, emulsion, or powder.

A typical probiotic ingredient is freeze-dried powder containing typically 10¹⁰-10¹² viable probiotic bacterial cells per gram. In addition it normally contains freeze drying carriers such as skim milk, short sugars (oligosaccharides such as sucrose or trehalose). Alternatively, the culture preparation can be encapsulated by using e.g. alginate, starch, xanthan as a carrier. A typical probiotic supplement or capsule preparation contains approximately 10⁹-10¹¹ viable probiotic bacterial cells per capsule as a single strain or multi-strain combination.

A typical probiotic food product, which can be among others fermented milk product or juice, contains approximately 10⁹-10¹¹ viable probiotic bacterial cells per daily dose. Probiotics are incorporated in the product as a probiotic ingredient (frozen pellets or freeze dried powder) or they are cultured in the product during fermentation.

The invention will be described in more detail by means of the following examples. The examples are not to be construed to limit the claims in any manner whatsoever.

### EXAMPLES

Materials and methods

The materials and methods described herein are common to examples 1 to 5.

59 healthy adult volunteers (52 females and 7 males) were recruited to the study. Both faecal and blood samples were collected from 59 volunteers. The age of the volunteers ranged from 31 to 61 and was in average 45 years.

Faecal samples were frozen within 5 hours from defecation. DNA from 0.3 g of faecal material was extracted by using the FASTDNA® SPIN KIT FOR SOIL (Qbiogene).

PCR-DGGE methods were optimised to detect dominant eubacteria (universal group), Eubacterium rectale-Clostridium coccoides (EREC) group, Bacteroides fragilis group, Clostridium leptum group. Partial eubacterial 16S rRNA gene was amplified by PCR with group specific primers (shown in table 1). Amplified PCR fragments were separated in 8% DGGE gel with denaturing gradient ranging from 45% to 60%. DGGE gels were run at 70 V for 960 mins.

DGGE gels were stained with SYRBSafe for 30 mins and documented with SafeImager Bluelight table (Invitrogen) and AplhaImager HP (Kodak) imaging system.

Digitalised DGGE gel images were imported to the Bionumerics-program version 5.0 (Applied Maths) for normalisation and band detection. The bands were normalised in relation to a marker sample specific for the said bacterial groups. Band search and bandmatching was performed as implemented in the Bionumerics. Bands and bandmatching were manually checked and corrected. Principal component analysis was calculated in Bionumerics. Other statistical analyses (Anova, Kruskal-Wallis test and Fisher exact test) were computed with statistical programming language R, version 2.8.1.

The bands were excised from DGGE gels. DNA fragments from bands were eluted by incubating the gel slices in 50 μl of sterile H₂O at +4° C. overnight. The correct positions and purity of the bands were checked for each excised bands by amplifying DNA in bands and re-running the amplified fragments along with the original samples in DGGE. Bands which produced single bands only and were in the correct position in the gels were sequenced. The sequences were trimmed, and manually checked and aligned by ClustalW. The closest relatives of the sequences were searched using Blast and NCBI nr database. Distance matrix of the aligned sequences was used to compare the similarity of the sequences.

TABLE 1
Primers and their sequences used in this study

| Target group | Primer | Sequence* | Reference** |
|---|---|---|---|
| Universal | U-968-F-GC | GC glamp1-AACGCGAAGAACCTTA | Nübel et al. 1996 |
| Universal | U-1401-R | CGGTGTGTACAAGACCC | Nübel et al. 1996 |
| Lactobacillus | Lac1 | AGCAGTAGGGAATCTTCCA | Walter et al.. 2001 |
| Lactobacillus | Lac2GC | GC glamp2-ATTYCACCGCTACACATG | Walter et al.. 2001 |
| EREC | CcocF | AAATGACGGTACCTGACTAA | Matsuki et al. 2002 |
| EREC | CcocR-GC | GC glamp1-CTTTGAGTTTCATTCTTGCGAA | Maukonen et al. 2006 |
| B. fragilis | BfraF | ATAGCCTTTCGAAAGRAAGAT | Matsuki et al. 2002 |
| B. fragilis | BfraR + GC | GC glamp1-CCAGTATCAACTGCAATTTTA | Matsuki et al. 2002 |
| C. leptum | Clept-F | GCACAAGCAGTGGAGT | Matsuki et al. 2004 |
| C. leptum | CleptR3-GC | GC glamp1-CTTCCTCCGTTTTGTCAA | Matsuki et al. 2004 |

*GC-glamp1 sequence: CGCCCGGGGCGCGCCCCGGGCGGGGCGGGGGCACGGGGGG
GC glamp2 sequence: CGCCCGCCGCGCCCCGCGCCCGGCCCGCCGCCCCCGCCCC
**References: Nübel et al. 1996 J Bacteriol. 178: 5636-43. Walter et al. 2001 Appl Environ Microbiol. 67: 2578-2585. Matsuki 2002 Appl Environ Microbiol. 68: 5445-51. Matsuki 2004 Appl Environ Microbiol. 70: 7220-8. Maukonen 2006. FEMS Microbiol Ecol. 58: 517-28.

TABLE 2
The optimised DGGE gel gradients, electrophoresis running conditions for
the each studied bacterial group and strains used in the standards

| | | | Electrophoretic running | |
|---|---|---|---|---|
| Bacterial | | DGGE gel | conditions in Dcode | |
| group | primers* | gradient | system (Bio-Rad) | Strains in standard |
| Universal | U968F-GC, | 38-60% | 70 V, 960 mins | A. cacae DSM 14662 |
| | U1401R | | | C. perfringens DSM 756 |
| | | | | E. ramulus DSM 15687 |
| | | | | F. prausnitzii DSM 17677 |
| | | | | E. coli DSM 30083 |
| | | | | L. rhamnosus DSM 96666 |
| | | | | P. melaninogenica DSM 7089 |
| Bifido- | Bif164F, | 45-60% | 70 V, 960 mins | B. adolescentis DSM 981074 |
| bacterium | Bif662R- | | | B. angulatum DSM 20098 |
| | GC | | | B. longum DSM 96664 |
| | | | | B. catenulatum DSM 16992 |
| | | | | B. lactis DSM 97847 |
| Lacto- | Lac1, | 38-55% | 70 V, 960 mins | L. plantarum E-79098 |
| bacillus | Lac2-GC | | | L. cellubiosis E-98167 |
| | | | | L. reuterii E-92142 |
| | | | | L. paracasei E-93490 |
| B. fragilis | BfraF, | 30-45% | 70 V, 960 mins. | B. caccae DSM 19024 |
| | BfraR-GC | | | B. uniformis DSM 6597 |
| | | | | B. eggerthii DSM 20697 |
| EREC | CcocF, | 40-58% | 70 V, 960 mins | L. multipara DSM 3073 |
| | CcocR-GC | | | A. cacae DSM 14662 |
| | | | | D. longicatena DSM 13814 |
| | | | | R. productus DSM 2950 |
| C. leptum | CleptF, | 30-53% | 70 V, 960 mins | F. prausnitzii DSM 17677 |
| | CleptR3- | | | C. methylpentosum DSM 5476 |
| | GC | | | R. albus DSM 20455 |
| | | | | C. leptum DSM 753 |
| | | | | E. siraeum DSM 15702 |
| | | | | C. viridae DSM 6836 |

*Primer sequences are in Table 2

### Example 1

Secretor status was determined from the blood samples by using an agglutination assay. Secretor status was determined from 59 individual and 48 were secretors and seven were non-secretors. The secretor status of four samples could not be determined; they were excluded from the further analyses.

### Example 2

In universal DGGE analysis of dominant intestinal bacteria, several genotypes occured statistically significantly more often or with a higher intensity in the non-secretor samples than in the secretor samples. All genotypes were 2 to 3.6 times more frequently detected in the non-secretor in comparison to secretor samples. The genotypes can be identified by the band positions on universal DGGE gel corresponding the band positions 25.30%, 26.40%, 50.40% and 56.80%. The band positions, genotypes, which differed between non-secretor and secretor individuals and their detection frequencies, are shown in Table 3.

TABLE 3
Statistically significant differences on band intensities between
non-secretor (NSS) and secretor (SS) samples as determined by universal-
DGGE (n = 55, NSS = 7, SS = 48). Statistical tests, ANOVA (ANO) and
Kruskal-Wallis (KW) were based on band intensity matrix and Fisher's
exact test (F) was based on presence/absence-matrix of the bands

| | | | | | | Mean |
|---|---|---|---|---|---|---|
| | | | | | | band |
| | | | | number | number | intensity |
| | | | | in NSS | in SS | in NSS/ |
| Genotype | Test | p-value | # of hits | (%) | (% | in SS |
| 25.30% | ANO/ | 0.03/0.05 | 18 (31) | 4 (57) | 14 (29) | 13/10 |
| | KW | | | | | |
| 26.40% | ANO/ | 0.002/0.02 | 4 (7) | 1 (14) | 3 (6) | 22/8 |
| | KW | | | | | |
| 50.40% | ANO | 0.03 | 6 (10) | 2 (29) | 4 (8) | 18/10 |
| 56.80% | KW/F | 0.006/0.01 | 10 (17) | 4 (57) | 6 (12) | 17/25 |

### Example 3

A genotype belonging to Eubacterium rectale-Clostridium coccoides-group (EREC) and corresponding band position 60.0% in EREC-DGGE gels was clearly more common in non-secretor than in secretor samples. The genotype was more than seven times more common in the samples from non-secretor individuals than in the samples of secretor individuals. The results are shown in Table 4.

TABLE 4
Statistically significant differences on band intensities between
non-secretor (NSS) and secretor (SS) samples as determined by EREC-
DGGE (n = 55, NSS = 7, SS = 48). Statistical tests, ANOVA (ANO) and
Kruskal-Wallis (KW) were based on band intensity matrix and Fisher's
exact test (F) was based on presence/absence-matrix of the bands

| | | | | | | Mean |
|---|---|---|---|---|---|---|
| | | | | | | band |
| | | p-value | | number | number | intensity |
| | | (ANO/ | # of | in NSS | in SS | in NSS/ |
| Genotype | Test | KW/F) | hits | (%) | (%) | in SS |
| 60.00% | ANO/ | 0.00002/ | 6 (10) | 3 (43) | 3 (6) | 30/11 |
| | KW/F | 0.0006/ | | | | |
| | | 0.04 | | | | |

### Example 4

Five genotypes of Bacteroides fragilis group were statistically significantly more common or more abundant in the non-secretor samples than in secretor samples. The genotype band position 23.80, as indicated by the controls, referred to Bacteroides uniformis strain DSM6597; this genotype was three times more common in the non-secretor samples than in the secretor samples. Other genotypes corresponded band positions 4.80%, 10.20%, 38.70%, and 41.10%. These band positions were also three times more commonly detected in the non-secretor than in secretor samples, except genotypes related to band positions 10.20% and 38.70%. Band positions 10.20% and 38.70% were equally common in the non-secretor and secretor samples, but the band intensity (i.e. abundance) was over two times higher in the non-secretor than in secretor samples. The results are shown in Table 5.

TABLE 5
Statistically significant differences on band intensities between
non-secretor and secretor samples as determined by B. fragilis group
DGGE (n = 55, NSS = 7, SS = 48). Statistical tests, ANOVA (ANO) and
Kruskal-Wallis (KW) were based on the band intensity matrix and
Fisher's exact test (F) was based on presence/absence-matrix of the bands

| | | p-value | | number | number | Mean band |
|---|---|---|---|---|---|---|
| | | (ANO/ | | in NSS | in SS | intensity |
| Genotype | test | KW/F) | # of hits | (%) | (%) | in NSS/in SS |
| 4.80% | KW | 0.04 | 6 (10) | 2 (29) | 4 (8) | 54/61 |
| 10.20% | ANO | 0.004 | 29 (49) | 4 (57) | 25 (52) | 93/35 |
| 23.80% | ANO/ | 0.0004/ | 13 (22) | 4 (57) | 9 (19) | 62/32 |
| | KW/F | 0.005/ | | | | |
| | | 0.03 | | | | |
| 38.70% | ANO | 0.02 | 24 (41) | 3 (43) | 21 (44) | 96/16 |
| 41.10% | ANO | 0.007 | 7 (12) | 2 (29) | 5 (10) | 53/39 |

### Example 5

Seven genotypes belonging to Clostridium leptum group were more common or abundant in the non-secretor samples than in secretor samples. The band positions corresponding these genotypes are listed in Table 6. The genotype in band position 36.10% was slightly more common in the non-secretors in comparison to the secretors, but this genotype was 3.8 times more abundant as measured by band intensity in the non-secretors. The results are shown in table 6.

TABLE 6
Statistically significant differences on band intensities between
non-secretor and secretor samples as determined by C. leptum DGGE
(n = 55, NSS = 7, SS = 48). Statistical tests, ANOVA (ANO) and
Kruskal-Wallis (KW) were based on band intensity matrix and Fisher's
exact test (F) was based on presence/absence-matrix of the bands

| | | | | | | Mean |
|---|---|---|---|---|---|---|
| | | | | | | band |
| | | p-value | | number | number | intensity |
| | | (ANO/ | # of | in NSS | in | in NSS/ |
| Genotype | test | KW) | hits | (%) | SS (%) | inSS |
| 32.80% | KW | 0.003 | 6 (10) | 3 (43) | 3 (6) | 15/25 |
| 36.10% | ANO | 0.03 | 7 (10) | 1 (14) | 5 (10) | 54/11 |
| 43.00% | ANO | 0.007 | 16 (27) | 3 (43) | 13 (27) | 95/35 |
| 73.30% | ANO | 0.001 | 14 (24) | 3 (43) | 11 (23) | 25/19 |
| 79.10% | ANO | 0.01 | 6 (10) | 2 (29) | 4 (8) | 52/30 |
| 85.00% | ANO/ | 0.007/ | 15 (25) | 5 (71) | 10 (21) | 25/20 |
| | KW | 0.005 | | | | |
| 91.80% | ANO/ | 0.0008/ | 8 (14) | 3 (43) | 5 (10) | 52/15 |
| | KW | 0.01 | | | | |

### Example 6

58 healthy adult volunteers were recruited to the study. Both faecal and blood samples were collected. The age of the volunteers ranged from 31 to 61 and was in average 45 years.

Secretor status was determined from blood samples by using the standard in-house blood grouping protocols of Finnish Red Cross Blood Service, Helsinki Finland. Fourty-nine samples were found to be secretors and six were non-secretors. For 3 samples, secretor status could not be accurately determined serologically from the blood sample.

Faecal samples were frozen within 5 hours from defecation. DNA from 0.3 g of faecal material was extracted by using the FASTDNA® SPIN KIT FOR SOIL (Qbiogene).

PCR-DGGE method was optimised for Lactobacillus-group. Partial eubacterial 16S rRNA gene was amplified by PCR with the group specific primers shown in Table 1. Amplified PCR fragments were separated in 8% DGGE gel with denaturing gradient ranging from 45% to 60%. DGGE gels were run at 70 V for 960 mins. DGGE gels were stained with SYRBSafe for 30 mins and documented with SafeImager Bluelight table (Invitrogen) and AplhaImager HP (Kodak) imaging system.

Digitalised DGGE gel images were imported to the Bionumerics-program version 5.0 (Applied Maths) for normalisation and band detection. Bands were normalised with marker sample specific for above mentioned bacterial groups were constructed from strains. Band search and bandmatching was performed as implemented in Bionumerics. Bands and bandmatching were manually checked and corrected. Principal component analysis was calculated in Bionumerics. Other statistical analysis were computed with statistical programming language R, version 2.8.1.

The bands were excised from DGGE gels. DNA fragments from bands was eluted by incubating the gel slices in 50 μl sterile H₂O at +4° C. overnight. The correct positions and purity of the bands were checked for each excised bands by amplifying DNA in bands and running the amplified fragments along the original samples in DGGE. Bands, which only produced single bands and were in the correct position in the gels, were sequenced. The sequences were trimmed, and manually checked and aligned by ClustalW. The closest relatives of the sequences were searched using Blast and NCBI nr database. Distance matrix of the aligned sequences was used to compare the similarity of the sequences.

TABLE 1
Primers and their sequences used in this study.

| Target group | Primer | sequences | Reference |
|---|---|---|---|
| Lactobacillus | Lac1 | AGCAGTAGGGAATCTTCCA | Walter et al. 2001** |
| Lactobacillus | Lac2GC | GC glamp2*-ATTYCACCGCTACACATG | Walter et al. 2001 |

*GC glamp 2 sequence: CGCCCGCCGCGCCCCGCGCCCGGCCCGCCGCCCCCGCCCC
**Walter et al. 2001, Appl Environ Microbiol. 67: 2578-2

### Results

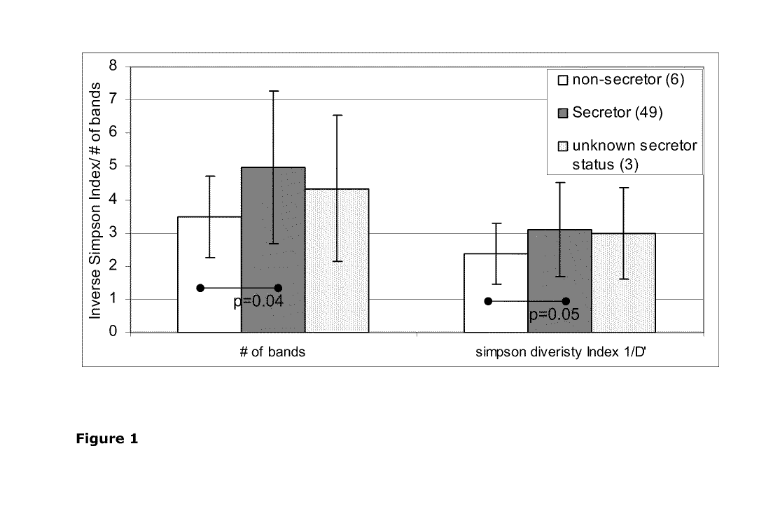

The richness, i.e. the number of bands or genotypes detected and the diversity in Lactobacillus-DGGE differed statistically significantly between the non-secretor and secretor samples. The non-secretor samples had a lower richness than secretor samples (p=0.04). Moreover, the diversity of Lactobacillus was lowered in the non-secretor samples as compared to the secretors (p=0.05; FIG. 1).

1. A microbial composition characterized in that it is tailored based on the bacterial genotype composition typical to individuals with non-secretor blood group phenotype.

2. A microbial composition characterized in that it is tailored based on the bacterial genotype composition typical to individuals with secretor blood group phenotype.

3. The microbial composition according to claim 1, comprising at least one strain having any one or more of the following bacterial genotypes
a) band position 25.30%, 26.40%, 50.40% or 56.80% as defined by universal-DGGE analysis; or
b) band position 60.0% as defined by Eubacterium rectale-Clostridium coccoides-group (EREC)-DGGE analysis; or
c) band position 4.80%, 10.20%, 23.80%, 38.70%, or 41.10% as defined by Bacteroides-DGGE analysis; or
d) band position 32.80%, 36.10%, 43.00%, 73.30%, 79.10%, 85.00%, or 91.80% as defined by Clostridium leptum-DGGE analysis.

4. The microbial composition according to claim 1 wherein the composition further comprises at least one prebiotic agent.

5. A method of tailoring a microbial composition based on the spectrum of microbes found from the intestine of at least one individual with non-secretor blood group phenotype.

6. A method of tailoring a microbial composition based on the spectrum of microbes found from the intestine of at least one individual with secretor blood group phenotype.

7. Use of the secretor/non-secretor blood group status of an individual in assessing the need for optimized microbial supplementation.

8. Use of secretor/non-secretor blood group status of an individual to predict the microbial composition of the gut microbiota of the individual.

9. The use according to claim 8 characterised in that predicted microbial composition is related to at least one of the bacterial group of the list: Bacteroides fragilis group, Clostridium leptum group, and/or Eubacterium rectale-Clostridium coccoides-group.

10. A method for determination of the balance of gut microbiota of an individual, comprising:
determining a secretor/non-secretor genotype of an individual from a sample;
determining a composition of gut microbiota of the individual from a sample; and
comparing the composition of the gut microbiota of the individual to the typical composition of gut microbiota according to the secretor/non-secretor genotype.

11. A use of the secretor/non-secretor blood group status of an individual in estimating a dose of microbial supplementation needed for a desired effect.

12. A use of the secretor/non-secretor status of an individual to augment the stabilisation of mucosal microbiota in disorders related to, or after treatments leading to unbalance of mucosal microbiota.

13. A method for treating disorders or diseases related unbalanced mucosal microbiota in an individual comprising administering to the individual a therapeutically effective amount of the microbial composition of claim 1.

14. A method for treating disorders or diseases having FUT2 gene as a susceptible factor in an individual comprising administering to the individual a therapeutically effective amount of the microbial composition of claim 1.

15. A method for treating inflammatory bowel disease, urogenital infection and/or low levels of vitamin B12 in an individual comprising administering to the individual a therapeutically effective amount of the microbial composition of claim 1.
